(19) United States
(12) Patent Application Publication
(10) Pub. No.: US 2019/0209189 A1
(43) Pub. Date: Jul. 11, 2019

(54) THROMBUS RETRIEVAL STENTS AND METHODS OF USING FOR TREATMENT OF ISCHEMIC STROKE

(71) Applicant: Mayank GOYAL, Calgary (CA)

(72) Inventor: Mayank GOYAL, Calgary (CA)

(21) Appl. No.: 16/071,124

(22) PCT Filed: Jan. 24, 2018
(86) PCT No.: PCT/CA2018/050084

§ 371 Date: Jul. 19, 2018

Related U.S. Application Data
(60) Provisional application No. 62/450,842, filed on Jan. 26, 2017.

(51) Int. Cl. A61B 17/221

(52) U.S. Cl. CPC A61B 17/221; A61M 2025/0042; A61B 2017/2212

(57) ABSTRACT

The invention describes systems and methods for retrieving blood clots (thrombi) from patients undergoing endovascular/neurointervention procedures following ischemic stroke are described. More specifically, blood clot retrieval devices for deployment into a patient's vasculature from a catheter that are effective in ensnaring and removing fibrin rich thrombi are described as well as methods of utilizing these devices.

### FIELD OF THE INVENTION

The invention describes systems and methods for retrieving blood clots (thrombi) from patients undergoing endovascular/neurointervention procedures following ischemic stroke. More specifically, clot retrieval devices effective in ensnaring and removing fibrin rich thrombi are described as well as methods of utilizing these devices.

### BACKGROUND OF THE INVENTION

The human body has an extensive network of blood vessels including both the venous and arterial systems for circulating blood throughout the body. The occurrence and/or development of restrictions to flow within the circulatory system can result in serious medical conditions, the most significant being myocardial infarction and ischemic stroke. The treatment of both conditions (and others involving the circulatory system) continues to evolve with many new techniques and equipment being utilized to effect treatment.

In recent years, a variety of traumatic surgical procedures have been replaced with procedures that involve the use of one or more catheters being advanced through the vascular system of the body to gain access to diagnose and/or treat issues involving the vasculature of a particular organ. For example, ischemic strokes caused by blood clot blockages in the brain, coronary artery blockages within the heart and various heart defects may be treated by advancing catheters to the affected site whence various procedures can be initiated to treat the problem. Stents having various structural and functional properties can be positioned and deployed at a location where intervention is required wherein the specific structure of the stent can allow the treatment of a medical problem. Catheter procedures are also undertaken in other parts of the body including the leg vessels and renal arteries and other complex percutaneous procedures including treatment of valvular heart disease, aortic dissections, dysrhythmias, and management of shunts for dialysis patients can also be performed using catheter systems. Further, complex aneurysms in the brain and other locations are increasingly being treated through a percutaneous endovascular route.

It is known that when a patient experiences a significant ischemic stroke event, those portions of the brain distal to the occlusion that experience a dramatic reduction in blood supply will affect the functioning of large regions of neurons. This reduction in blood supply may cause the patient to become symptomatic, cause the death of regions of the brain and/or put regions of the brain at the risk of dying if not treated quickly. Depending on the location and size of the occlusion will result in a wide range of symptoms in the patient and depending on the severity will ultimately determine how a physician may choose to intervene or not.

Time delays in effecting treatment will typically result in the death of a greater number of neurons. Table 1 shows that in the specific case of acute ischemic stroke, the pace or rate of neural circuitry loss in a typical large vessel supratentorial acute ischemic stroke can be very rapid.

TABLE 1
Estimated Pace of Neural Circuitry Loss in Typical Large Vessel,
Supratentorial Acute Ischemic Stroke
Estimated Pace of Neural Circuitry Loss in Typical Large Vessel,
Supratentorial Acute Ischemic Stroke

| | Neurons | Synapses | Myelinated | Accelerated |
|---|---|---|---|---|
| | Lost | Lost | Fibers Lost | Aging |
| Per | 1.2 billion | 8.3 trillion | 7140 km/4470 miles | 36 yrs |
| Stroke | | | | |
| Per | 120 billion | 830 billion | 714 km/447 miles | 3.6 yrs |
| Hour | | | | |
| Per | 1.9 million | 14 billion | 12 km/7.5 miles | 3.1 weeks |
| Minute | | | | |
| Per | 32,000 | 230 million | 200 meters/218 | 8.7 hours |
| Second | | | yards | |

The numbers presented above represent an average with it also being known that there is a high degree of variability in the above numbers generally depending on the available blood supply to the ischemic region through collateral channels. However, and importantly, delays in making a decision in the order of only a few minutes can have a significant impact on neural circuitry loss and ultimately patient outcome. Further, even slight variations in blood supply can tip the balance and dramatically further increase the rate of cell death if blood supply is reduced or, alternatively prevent neural cell death if blood supply is restored quickly.

The recent paper “Analysis of Workflow and Time to Treatment and the Effects on Outcome in Endovascular Treatment of Acute Ischemic Stroke: Results from the SWIFT PRIME Randomized Controlled Trial” (Radiology, accepted for publication Feb. 24, 2016), and incorporated herein by reference, quantitatively shows that there is a definitive improvement in patient outcome through fast reperfusion. In particular, this study concluded that “aggressive time goals may have contributed to efficient workflow environments”. Further, the study quantifies inter alia that functional independence of a patient was significantly higher when treated quickly (i.e within 2.5 hours of stroke onset).

Importantly, it is now known that efficient workflows during a recanalization procedure (of which the effectiveness of a stent is important) provided better outcomes.

In diagnosing and treating ischemic stroke, it is important for the physician to know where the vessel occlusion is, how big the occlusion is, where any dead brain tissue (termed “core”) is and, how big and where is the brain tissue that may have been affected by the ischemic event but that may potentially be saved (termed “penumbra”).

The penumbra is tissue around the ischemic event that can potentially stay alive for a number of hours after the event by the perfusion of this tissue by collateral arteries. The collateral arteries may provide sufficient oxygen, nutrients and/or flushing to the penumbra tissue to prevent this tissue from dying for a period of time.

When responding to acute ischemic stroke, endovascular treatment of acute ischemic stroke due to large vessel occlusion in the anterior circulation is now the standard of care for patients under certain criteria. That is, patients exhibiting particular symptoms (i.e stroke symptoms of a particular severity) will benefit from early and rapid endovascular intervention to open occluded blood vessels. During various endovascular treatments, a surgeon will advance clot-retrieval and/or clot-suction devices into the brain's vasculature to the location of the clot where the clot is either withdrawn and/or aspirated from the clot site.

There are many anatomical and situational considerations that can affect the severity and ultimately treatment of ischemic stroke. Importantly, as described above, while a blood clot may severely affect blood flow to the ischemic area, some blood flow may get to the ischemic area if collateral arteries are functioning to at least partially perfuse the affected area.

The most common large vessel occlusion that is treated by endovascular techniques is the M1 segment of the middle cerebral artery (MCA). When a patient has an M1 occlusion, the territory supplied by the M1 receives a dramatic reduction in blood supply. As a consequence distal neurons don't function well and the patient becomes symptomatic. Preferably, there is some blood flow that manages to get to the ischemic territory through collaterals which may decrease the rate of neuronal death. Generally, in this case, the collaterals are the connections between the distal most branches of the anterior cerebral artery and the middle cerebral artery (or the posterior cerebral artery and the middle cerebral artery).

In different patients, collaterals are highly variable and there are a number of factors at play which are not fully understood. Some of these factors are genetic in nature but conditions such as hypertension and diabetes (and other poorly understood factors) may also reduce the efficacy of collaterals in different patients.

Regardless of the patient's anatomy, the maintenance of collateral blood flow is critical to keep the brain alive until the time the occluded vessel can be recanalized and blood flow re-established.

Recanalization procedures utilize a wide range of equipment and techniques to access a clot and effect its removal. Generally, the endovascular surgeon will have a number of tools at their disposal including a wide range of guide catheters, microcatheters, microwires, stents and other tools that individually have properties, features and functions that are effective for different procedures and patient presentations.

When an endovascular surgeon deploys a stent to retrieve a clot, the stent is generally conveyed to the clot within a microcatheter in a compressed state. The typical modern stent is a fine mesh of wires that once expanded form a small network of criss-crossing wires that upon deployment penetrate the surface of the clot and otherwise engage with the clot to allow the clot to be drawn proximally from the occlusion site and removed from the body. Generally, engagement of the wires with the clot requires that the wires penetrate the surface of the clot in a manner that sufficient friction and/or interfacial forces between the clot and wires exist to wholly and fully allow the dot to be withdrawn. Generally, the mesh of wires can be open or closed cell designs where most closed cell design stents will foreshorten as they are deployed.

During a typical dot retrieval procedure, 75-85% of clots, once accessed, will engage with the stent and can be removed. However, for the remaining 15-25% of clots, the properties of the clot prevent effective penetration of the wires to within the clot with the result being that when the surgeon attempts to withdraw the stent, the wires of the stent pass over the outer surface of the clot and the clot remains at the occlusion site.

Generally, the reason that such clots cannot fully engage with the wires of the stent is that surface and/or density of the clot prevents penetration of the wire. More specifically, such clots may have a tougher or harder outer surface that may have formed as result of a number of factors including where the clot formed, its age, the patient's blood chemistry and other factors. Typically, these clots are characterized as having a higher fibrin content that affects the surface and internal density of the clot making it more difficult for the stent wires to penetrate.

Presently, there are limited tools or techniques allowing the surgeon to penetrate the surface of such clots if there is an initial failure with a regular stent. Using current tools, the surgeon will typically deploy a stent at the clot, wait a 1-3 minutes for the clot to engage with the wires of the stent and then gently pull to retrieve the clot. Generally, it is only after this technique is undertaken that the surgeon will determine that the clot failed to engage which is the signal that the clot may have a tougher surface and cannot be retrieved. In these cases, the surgeon may be left with no option but to leave the clot where it is.

Various attempts have been made to design stents that have the capability to penetrate the surfaces of tougher clots. The main problem of designing a stent that may be more aggressive in its structure, for example, a stent having sharper surfaces that can penetrate the clot, is the significant risk of damaging vessel intima. In other words, while a stent may be provided with features that can penetrate a tough clot, those features may equally penetrate the arterial intima such that either upon deployment of the stent and/or upon proximal movement of the stent, the arterial intima is scratched and/or cut by the stent. Removal of a clot but with damage to the vessels will create substantial problems that must be avoided.

Moreover, when withdrawing the stent, the stent will be subject to various turns and twists as it withdrawn from the occlusion site. For example, the tortuosity of the vessels will cause the stent to deform as it passes through a curve. As a result, the central axis of the stent will not be continuously aligned with the central axis of the vessel as it is being withdrawn. Thus, the outer wires of the stent do not inherently protect the central axis of the stent. Moreover, in some situations, a stent will also deform as it is being withdrawn particularly when it is being drawn around a tight corner which can result in the stent losing or releasing the clot.

It is also known that the degree of tortuosity within blood vessels increases with age due to multiple factors including atherosclerotic disease, loss of height of the spine, etc. With an aging population and improving technologies, more and more of these procedures are being done in older patients necessitating access despite the increased complexity of conducting procedures through tortuous vessels. As a result, there is a need for improved stents that are more capable of travelling through tighter curves and that are less likely to drop a clot as the clot is being withdrawn.

Accordingly, there has been a need for systems and methods that are more effective in the capture and removal of fibrin-rich clots that cannot be removed by existing retrieval systems. In particular, there has been a need for retrieval systems having effective surfaces that enable penetration of fibrin rich clots that increase the likelihood of capture within designs that do not significantly increase the risk of damaging vessel intima during deployment and retraction.

### SUMMARY OF THE INVENTION

In accordance with the invention, there is provided systems and methods for improving the efficiency of surgical procedures using catheter systems to move from an entry point to a location in the body where a treatment or diagnostic procedure may be completed.

In a first aspect, the invention provides a blood clot retrieving apparatus for deployment into a patient's vasculature from a catheter and for retrieving an intravascular blood clot from within the patient's vasculature comprising: a push wire; and an expandable wire frame operatively connected to the push wire adjacent a distal end of the push wire, the expandable wire frame having a plurality of expandable wires having first and second ends operatively connected to the push wire, the expandable wire frame expandable from a compressed position within the catheter to an uncompressed deployed position, wherein each expandable wire of the expandable wire frame define a proximally facing tip, each proximally facing tip having a retraction wire operatively connected between the proximally facing tip and the push wire, the retraction wire operable to cause retraction of the proximally facing tip towards the push wire.

In another embodiment, the apparatus includes a microcatheter operatively retaining the push wire and where the retraction wire is slidingly engaged with the push wire via a guide located on the push wire adjacent the proximally pointing tip and where the retraction wire is operable from outside a proximal end of the microcatheter to cause deflection of the proximally pointing tip towards the push wire.

In another embodiment, the apparatus includes a microcatheter operatively retaining the push wire and where the retraction wire is fixed to the push wire at a distance from the proximally pointing tip where extension of the microcatheter over the retraction wire causes deflection of the retraction wire to draw each proximally pointing tip towards the push wire.

In one embodiment, the microcatheter and retraction wire have scale markings at their respective proximal ends, the scale markings dimensioned to provide visual information to an operator to indicate the radial position of the proximally pointing tips.

In one embodiment, the microcatheter and push wire have scale markings at their respective proximal ends, the scale markings dimensioned to provide visual information to an operator to indicate the radial position of the proximally pointing tips.

In another aspect, the invention provides method of deploying a blood clot retrieving apparatus (BCRA) as described herein within a vasculature to effect removal of a blood clot comprising the steps of: a) advancing a compressed BCRA within a microcatheter to a position within the vasculature adjacent a blood clot; b) withdrawing the microcatheter relative to the BCRA to deploy the BCRA adjacent the blood clot; c) advancing the BCRA in a proximal direction to effect blood clot capture; d) activating the retraction wire to effect inward deflection of the proximally facing tips; and, e) withdrawing the BCRA and microcatheter in a proximal direction to remove the blood clot from the vasculature.

### BRIEF DESCRIPTION OF THE DRAWINGS

Various objects, features and advantages of the invention will be apparent from the following description of particular embodiments of the invention, as illustrated in the accompanying drawings. The drawings are not necessarily to scale, emphasis instead being placed upon illustrating the principles of various embodiments of the invention; however, the scale of the drawings may be relied upon for supporting the relative position of described components with respect to one another. Similar reference numerals indicate similar components.

FIG. 1 is a schematic view of an aortic arch and associated blood vessels.

FIG. 1A is a schematic sketch of a portion of brain vascular anatomy showing the ophthalmic artery (OA), intracranial internal carotid artery (IICA), anterior cerebral artery (ACA), M1 segment of the middle cerebral artery and M2 segment of the middle cerebral artery.

FIG. 1B is a schematic sketch as in FIG. 1A showing a clot retrieval device engaged with a clot during a clot removal procedure.

FIG. 1C is a schematic sketch as in FIG. 1A showing a clot retrieval device engaged with a clot during a clot removal procedure and showing how a clot retrieval may compress around tortuous curves. The clot is shown disengaging from the clot retrieval device.

FIG. 2 is a side view of a stent system (clot retrieval system-CRS) in accordance with one embodiment of the invention having two distally facing wire frames and one proximally facing wire frame.

FIG. 3 is a perspective view of a proximally facing wire frame in accordance with one embodiment of the invention.

FIG. 4 is a side view of a proximally facing wire frame in a partially collapsed state in accordance with one embodiment of the invention.

FIG. 5 is a side view of a proximally facing wire frame in an extended state in accordance with one embodiment of the invention.

FIG. 6 is a side view of a proximally facing wire frame in a partially collapsed state in accordance with one embodiment of the invention.

### DETAILED DESCRIPTION OF THE INVENTION

### Introduction

With reference to the figures, systems and methods for retrieving blood clots via endovascular intervention are described. More specifically, systems adapted for retaining clots and particularly fibrin rich clots for removal from a patient's vasculature following ischemic stroke are described.

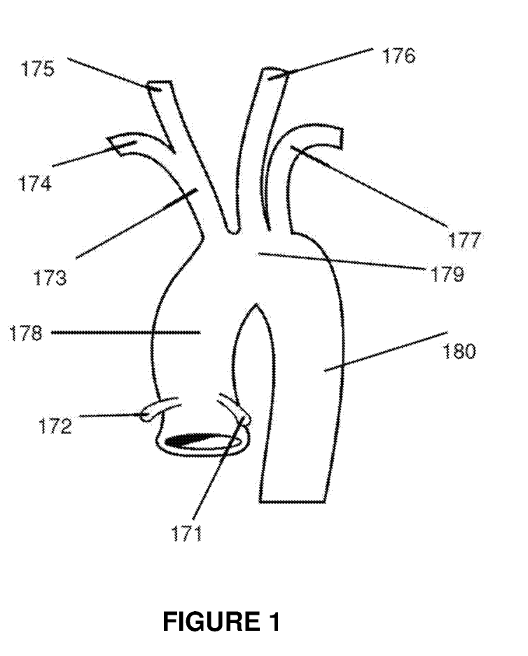

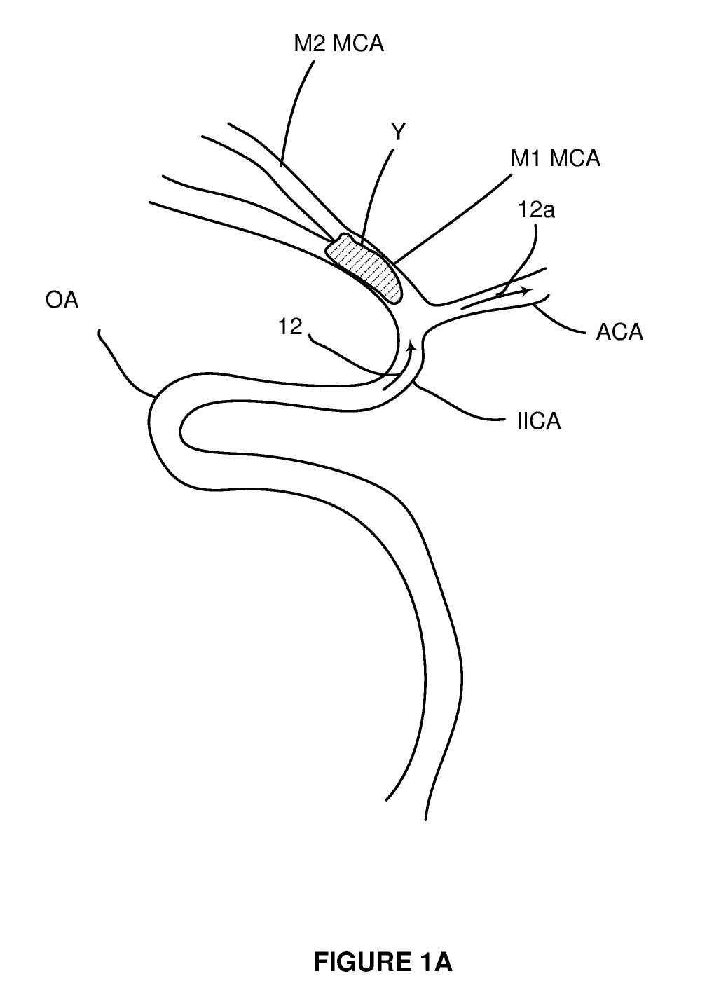

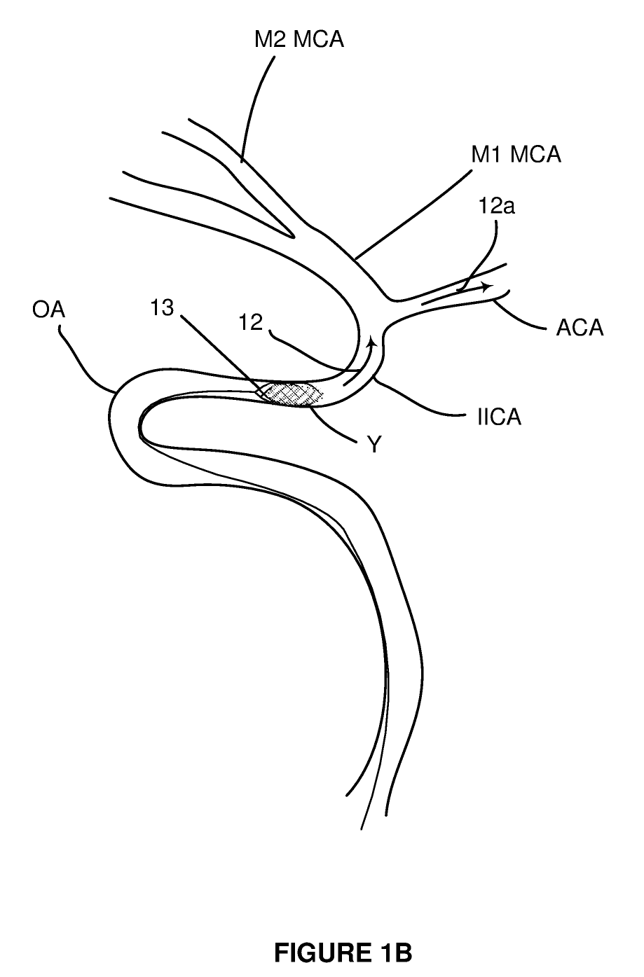

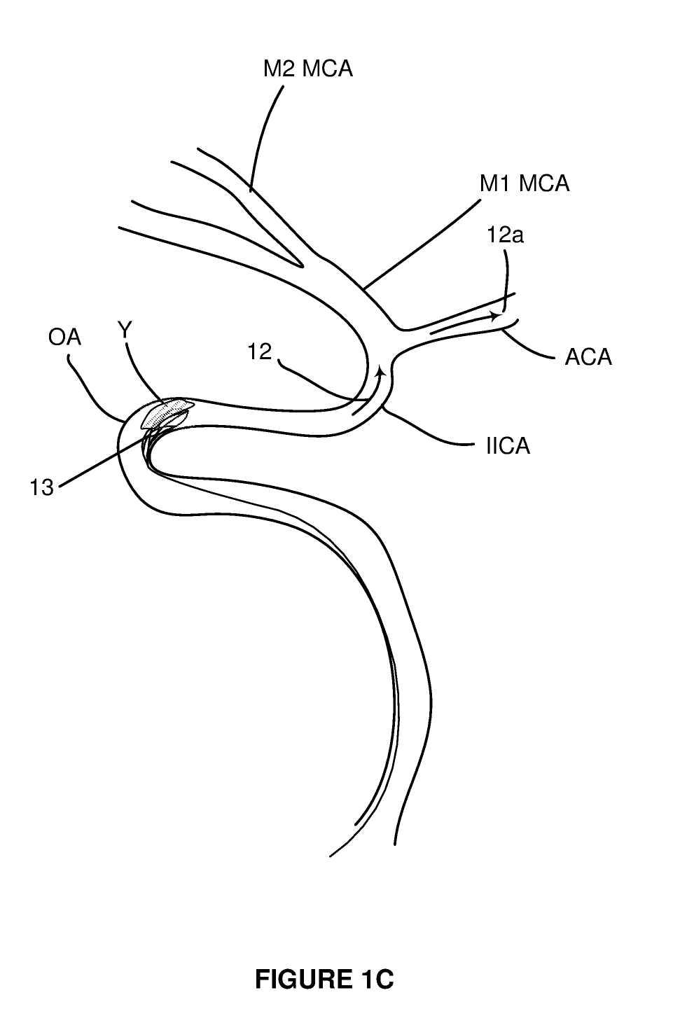

By way of background, FIG. 1 shows a typical aortic arch 179 and various connecting vessels in a human. The aortic arch 179 is connected to the ascending aorta 178 and the descending aorta 180. The ascending aorta is connected to the right and left coronary arteries 171, 172. The aortic arch is connected to the brachiocephalic artery 173 which splits into the right subclavian artery 174 and the right common carotid artery 175. Also connected to the aortic arch are the left common carotid artery and the left subclavian artery.

In a typical endovascular procedure utilizing one or more catheters to access the blood vessels in the head, the interventionist/surgeon typically navigates a catheter system up the descending aorta 180 from the femoral artery and into the aortic arch 179 and into the left common carotid artery 176. For the purposes of the description herein a “catheter system” implies various combinations of an inner guide wire (or microwire), outer catheter (or microcatheter), a distal access catheter (or a balloon guide catheter) and clot retrieval systems that may be advanced to the site of a clot. The various catheters are typically coaxial and can slide over or within the other although non-coaxial systems may also be used. In most procedures, the various components will be selectively moved through the patient's vasculature to a) gain access to the occlusion site and b) deploy a dot retrieval device to remove the clot.

Access to the clot (i.e. via antegrade or distal movement in direction of blood flow) is generally conducted by a combination of advancing a guide wire and advancing a microcatheter over the guide wire through the vasculature by twisting and turning the microcatheter and guide wire in order to direct the distal end of the guide wire and microcatheter into the appropriate vessel.

Clot retrieval devices are typically deployed through a microcatheter after a guide wire has been withdrawn.

Various procedures may also involve a tri-axial approach where the procedure includes advancing an outer catheter (e.g. a ‘distal access catheter’, ‘guide-catheter’ or ‘balloon guide catheter)’ to a position close to the clot and where the outer catheter is used after placement to rapidly advance a microcatheter and/or clot retrieval device to the occlusion site.

In general, it is known that the progression and movement of the various catheters through a patient's vasculature may be varied with the surgeon choosing or utilizing various techniques to advance these tools into a specific and desired location.

For the purposes of general description of the subject invention, it is assumed that the surgeon has advanced a larger guide catheter close to but proximal to the clot and a microwire and microcatheter at or beyond a clot. The microwire has been withdrawn allowing the surgeon to then advance a stent or clot retrieval device through the microcatheter and/or guide catheter where it is pushed out of the end of the microcatheter at or beyond the occlusion. As the stent moves beyond the end of the microcatheter, it expands outwardly and against the walls of the vessel. The surgeon may then simultaneously start to withdraw the microcatheter and stent where the expanded wires of the stent interact with the clot and entrap the clot within the wire cage of the stent. After a short period of time, if the clot has successfully engaged with the wire cage, the microcatheter and stent can be withdrawn into the guide catheter and then withdrawn from the body together with the clot.

FIG. 1A is a schematic diagram of brain vascular anatomy showing the intracranial internal carotid artery (IICA), anterior cerebral artery (ACA), M1 segment of the middle cerebral artery and M2 segment of the middle cerebral artery. A clot Y is shown within the M1 MCA with arrow 12 showing the direction of blood flow prior to any procedure. For the purposes of discussion, it is understood that blood flow 12a through the ACA is supporting collateral perfusion to affected areas of the brain. FIG. 1A also shows a tortuous region (eg. the ophthalmic artery (OA)) which is a region that can be difficult both to advance and withdraw catheter systems through. In some cases, due to the tortuosity of these vessels, as a surgeon is withdrawing a stent 13 that has been entangled with clot Y (FIG. 1B), the stent 13 may be flattened as it is drawn through a tortuous section resulting in the release or dropping of the dot Y (FIG. 1C) as the wires of the stent move with respect to one another.

Various aspects of the invention will now be described with reference to the figures. For the purposes of illustration, components depicted in the figures are not necessarily drawn to scale. Instead, emphasis is placed on highlighting the various contributions of the components to the functionality of various aspects of the invention. A number of possible alternative features are introduced during the course of this description. It is to be understood that, according to the knowledge and judgment of persons skilled in the art, such alternative features may be substituted in various combinations to arrive at different embodiments of the present invention.

As noted above, in a number of cases (approximately 15-25% of the time), the clot will fail to engage with the wire cage, as the wires of the cage fail to penetrate the surface of the clot. In these cases, as the surgeon tries to withdraw the stent, the wires of the wire cage will, for example, slip over and off the clot and the clot will remain in place.

Also, for the purposes of description, retrieval of a fibrin-rich clot is described where a surgeon has initially been unsuccessful retrieving the clot using a prior art stent. In these circumstances, after unsuccessfully withdrawing the clot, the stent is withdrawn into the microcatheter for removal from the occlusion site. Practically, in these situations, the surgeon will withdraw the stent into the microcatheter and then withdraw the microcatheter and stent proximally into a guide catheter thus allowing a new stent (as described below) to be inserted into the guide catheter and then quickly advanced back to the clot.

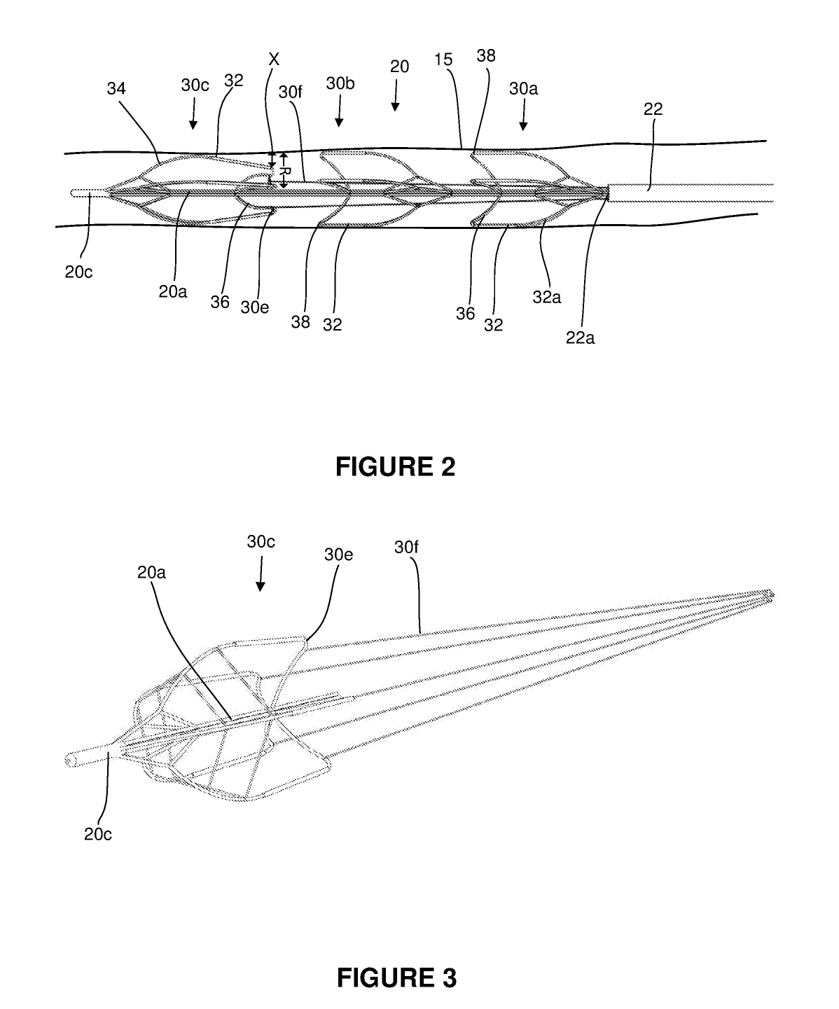

FIGS. 2-6 describe a blood clot removing apparatus (BCRA) or stent 20 having multiple wire frames 30a, 30b, 30c that may be deployed from a microcatheter (MC) 22 from a distal end 22a. Within this description, the wire frames are described as asymmetric meaning that each wire frame has a shape that is not symmetric along its length.

This application introduces the concept of distally facing wire frames and proximally facing wire frames which generally means that a wire frame has particular features at its proximal and distal ends that are different. In this description, a stent having two distally facing wire frames 30a, 30b and one proximally facing wire frame 30c are described, however this is not meant to be limiting in terms of the invention. A distally facing wire frame generally has surfaces that are favorable for proximal movement of the stent in a vasculature whereas a proximally facing wire frame has surfaces that when expanded are less favorable for proximal movement but are beneficial for tough clot capture.

Each of the wire frames are connected to a push wire 20a that provides a central axis to the wire frames. The wire frames are collapsible and moveable within a MC 22 such that when the MC and stent are positioned past and/or adjacent a clot, the MC can be withdrawn relative to push wire such that the wire frames emerge from the MC tip 22a into the vessel 15. As each of the wire frames have an inherent shape memory, these wire frames will expand to a pre-determined shape.

As noted, the distally facing wire frames 30a, 30b include surfaces that enable proximal movement of the CRS and that when in contact with a vessel are less likely damage the intima of the vessel due to the contact angle of such surfaces during movement. For example, the proximally facing surfaces 32a of the wire frame are shaped such that any contact with the intima will be substantially tangential or parallel to the vessel intima. The distally facing wire frames may include distally facing tips 38.

In contrast wire frame 30c will have proximally facing tips 30e that, when expanded and if withdrawn proximally have the potential to damage the intima particularly as such surfaces are drawn through curving vessels. Importantly, the tips 30e have a greater capability of penetrating a tough clot such that in the event that the more proximal wire frames are not successful in engaging a clot, the tips 30e may improve the likelihood of capture as the tips can impact the outer surface of the clot at a more normal angle to the clot surface which may be sufficient to penetrate the outer surface of the clot and cause entanglement.

However, as the tips 30e are radially distant from the push wire axis, proximal movement of the stent through the vessel, and in particular curved vessels could cause damage to the intima.

Accordingly, the tips 30e are provided with a retraction system that enables the surgeon to control the radial position R of the tips 30e and that enable them to be displaced a distance X during the procedure. Tips 38, which are distally facing, do not require a retraction system.

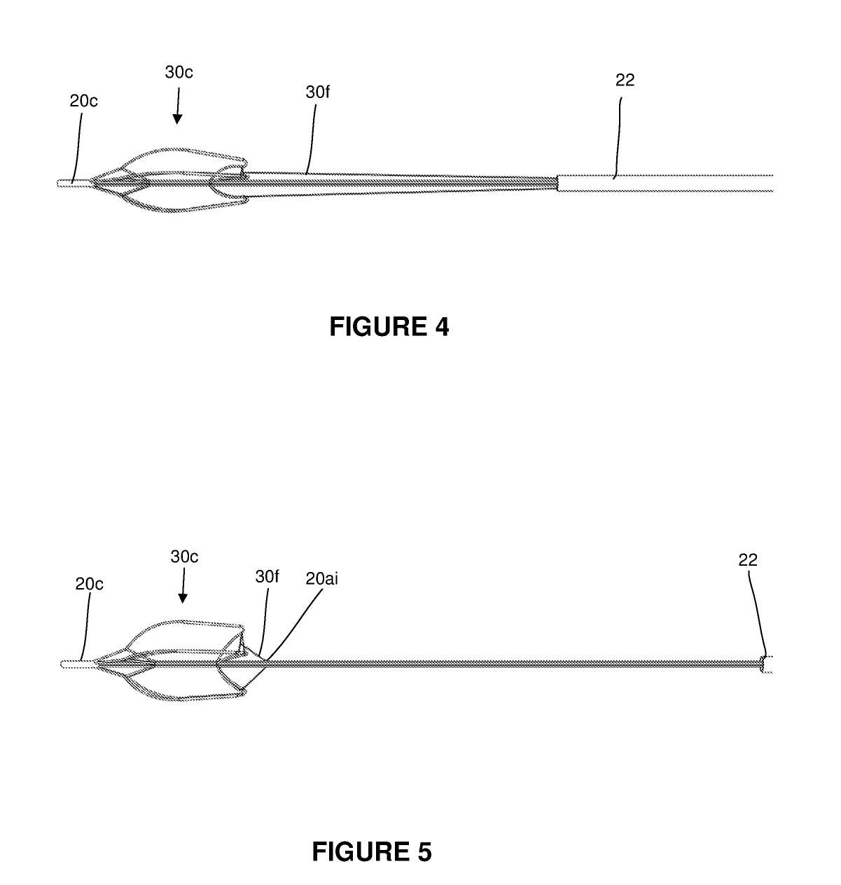

FIGS. 2-4 shown a first embodiment of a retraction system where FIG. 2 shows 2 distally facing wire frames and one proximally facing wire frame and FIGS. 3 and 4 show one proximally facing wire frame (in a fully extended and partially extended position respectively) where the distally facing wire frames are removed for clarity for the purposes of describing the retraction system.

As shown, each tip 30e is provided with a retraction wire 30f connected to a proximal location on the push wire 20a and each tip 30e. The retraction wires can be controlled by either moving the push wire relative to the MC such that the distal edge 22a of the MC applies a tangential force along the retraction wires thus creating an inward pulling force on the proximally pointing tips 30e as shown in FIGS. 2 and 4. In other words, as the MC 22 moves over the retraction wires towards distal end 20c thus shortening the distance between the MC tip 22a and the distal end 20c, the tips 30e are drawn inwardly. This allows the surgeon to adjust position of the tips and thus minimize the risk of the tips 30e from fully contacting the intima particularly as the stent removal process is initiated. For example, when the stent is deployed in an attempt to engage a tough clot, proximal movement of the stent a few millimeters may be sufficient to penetrate and entangle the clot without damaging the intima. However, full removal of the stent between 50 and 150 mm towards a guide catheter through narrow and tortuous vessels could result in damage. As such, tips 30e will preferably each have a rounded surface allowing relatively small proximal movements of the stent where the tips 30e will ride over the intima without damage.

It is important to note that the stents described herein would generally be used in procedures where other stents have not been successful and thus, would be considered a secondary or last choice of the surgeon to withdraw the clot. As such, while the surgical risk may be increased, the likelihood of success of a procedure to retrieve a tough clot is also increased.

Preferably, the retraction system will enable the tips 30e to be drawn inwardly a distance X such that the tips 30e are substantially contacting the push wire 30 when fully retracted.

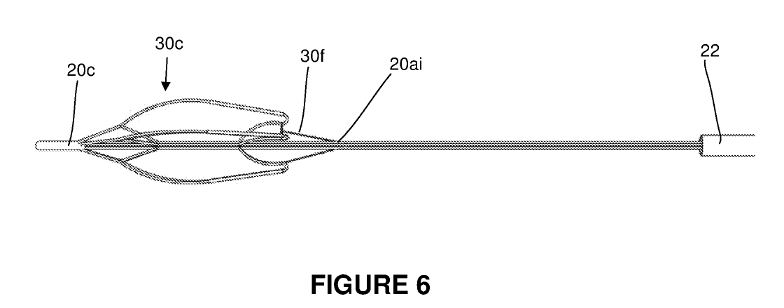

In another embodiment as shown in FIGS. 5 and 6, the retraction wires are drawn towards the push wire at a location closer to the wire cage 30c. In this case, the retraction wires may pass through a guide 20ai allowing the retraction to pass along or through the push wire such that by applying a proximal tension on the retraction wire from outside the MC, the proximally pointing tips are drawn towards the push wire.

The shape of each wire frame 30a, 30b and 30c may include features that promote extension and retraction of each frame from and into a MC. Generally, in the design shown each wire frame 30a, 30b and 30c has a section 32 that when fully expanded lies parallel to a vessel wall. Frames 30a and 30b have a proximally facing surface 32a tapering outwardly and distally from a push wire connection point whereas frame 30c has a distally facing surface 34 tapering outward and proximally from the push wire. Each wire frame will have a further wire 36 connecting either the proximally or distally facing tips to the push wire such that each wire frame is connected at both its proximal and distal ends to the push wire.

### Other Features

Preferably, the CRS will include markers on the proximal ends of the MC and push wires to provide the surgeon information with respect to the relative position of the MC and guide wire with respect to one another during a procedure. That is, the push wire and MC may be provided with proximal markers (i.e markers at the proximal ends) that show the relative position of the MC and either the guide wire or retraction wire relative to one another such that the surgeon can determine the degree of retraction of the tips 30e.

The design of a particular stent may or may not have wire frames 30a, 30b and may have wire frames having different features to those described above. While it will generally be desirable to have multiple wire frames along the push wire so as to increase the likelihood of clot capture, the system can be designed without additional wire frames.

### Typical Dimensions

For the purposes of illustration, Table 2 shows approximate dimensions of various components of the system.

TABLE 2
Typical Dimensions

| Component | Range (mm) | |
|---|---|---|
| Typical Vessel Diameter with Clot | 3-5 | mm |
| Guide Catheter OD | 2.5-3.5 | |
| Microcatheter OD | 0.5-0.7 | |
| Push Wire OD | 0.2-0.3 | |
| Wire of Wire Frame Diameter | 0.2-0.3 | |
| Wire Frame Length (per wire frame) | 5-10 | |

### Methods of Use

A surgeon may implement a number of different techniques to place and withdraw a CRS. As noted above, a CRS is typically deployed through a MC and withdrawn into a guide catheter. However, in various designs, the CRS may be fully withdrawn back into the MC. The surgeon will utilize a number of different push, pull and twisting techniques to properly place, deploy and withdraw a CRS during a procedure.

By being able to adjust the radial position of the proximally facing tips, the surgeon has a greater degree of control at the stent that can be utilized to effect removal of clots that otherwise may not be removal with present stents.

Although the present invention has been described and illustrated with respect to preferred embodiments and preferred uses thereof, it is not to be so limited since modifications and changes can be made therein which are within the full, intended scope of the invention as understood by those skilled in the art.

1. A blood clot retrieving apparatus (BCRA) for deployment into a patient's vasculature from a catheter and for retrieving an intravascular blood clot from within the patient's vasculature comprising:
a push wire;
an expandable wire frame operatively connected to the push wire adjacent a distal end of the push wire, the expandable wire frame having a plurality of expandable wires having first and second ends operatively connected to the push wire, the expandable wire frame expandable from a compressed position within the catheter to an uncompressed deployed position, wherein each expandable wire of the expandable wire frame define a proximally facing tip, each proximally facing tip having a retraction wire operatively connected between the proximally facing tip and the push wire, the retraction wire operable to cause retraction of the proximally facing tip towards the push wire.

2. The apparatus as in claim 1 further comprising a microcatheter operatively retaining the push wire and where the retraction wire is slidingly engaged with the push wire via a guide located on the push wire adjacent the proximally pointing tip and where the retraction wire is operable from outside a proximal end of the microcatheter to cause deflection of the proximally pointing tip towards the push wire.

3. The apparatus as in claim 1 further comprising a microcatheter operatively retaining the push wire and where the retraction wire is fixed to the push wire at a distance from the proximally pointing tip where extension of the microcatheter over the retraction wire causes deflection of the retraction wire to draw each proximally pointing tip towards the push wire.

4. The apparatus as in claim 2 where the microcatheter and retraction wire have scale markings at their respective proximal ends, the scale markings dimensioned to provide visual information to an operator to indicate the radial position of the proximally pointing tips.

5. The apparatus as in claim 3 where the microcatheter and push wire have scale markings at their respective proximal ends, the scale markings dimensioned to provide visual information to an operator to indicate the radial position of the proximally pointing tips.

6. A method of deploying a blood clot retrieving apparatus (BCRA) comprising a push wire and an expandable wire frame operatively connected to the push wire adjacent a distal end of the push wire, the expandable wire frame having a plurality of expandable wires having first and second ends operatively connected to the push wire, the expandable wire frame being expandable from a compressed position within the catheter to an uncompressed deployed position, wherein each expandable wire of the expandable wire frame defines a proximally facing tip, each proximally facing tip having a retraction wire operatively connected between the proximally facing tip and the push wire, the retraction wire being operable to cause retraction of the proximally facing tip towards the push wire within a vasculature to effect removal of a blood clot, the method comprising the steps of:
a. advancing a compressed BCRA within a microcatheter to a position within the vasculature adjacent a blood clot;
b. withdrawing the microcatheter relative to the BCRA to deploy the BCRA adjacent the blood clot;
c. advancing the BCRA in a proximal direction to effect blood clot capture;
d. activating the retraction wire to effect inward deflection of the proximally facing tips; and,
e. withdrawing the BCRA and microcatheter in a proximal direction to remove the blood clot from the vasculature.
